(19) United States
(12) Patent Application Publication
(10) Pub. No.: US 2010/0184068 A1
(43) Pub. Date: Jul. 22, 2010

(54) METHOD OF MEASURING REVERSE-TRANSCRIBED SINGLE STRANDED DNA, METHOD OF MEASURING REVERSE TRANSCRIPTASE ACTIVITY AND KIT FOR THE SAME

(75) Inventors: Joo-won Rhee, Seoul (KR); Sung-woo Hong, Gyeonggi-do (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/687,239

(22) Filed: Jan. 14, 2010

(30) Foreign Application Priority Data
Jan. 14, 2009 (KR) 10-2009-003043

(51) Int. Cl. C12Q 1/68

(52) U.S. Cl. 435/6

(57) ABSTRACT

Provided are a method of measuring a single stranded DNA reverse-transcribed from a RNA using RNaseH, RNaseT1, and RNase A, a method of measuring activity of a reverse transcriptase, and kits for the same.

### CROSS-REFERENCE TO RELATED APPLICATIONS

This application claims priority to Korean Patent Application No. 10-2009-0003043, filed on Jan. 14, 2009, and all the benefits accruing therefrom under 35 U.S.C. §119, the contents of which is incorporated herein in its entirety by reference.

### BACKGROUND

1. Field

One or more embodiments relate to a method of measuring a single stranded DNA reverse-transcribed from a RNA template, a method of measuring activity of a reverse transcriptase, and kits for the same.

2. Description of the Related Art

Reverse transcription assays are widely used for analysis of biological substances. One conventional method of measuring products of the reverse transcription includes measuring the concentration of products (ssDNA) of the reverse transcription by performing polymerase chain reaction (PCR). The ssDNA products synthesized from mature RNA using the enzyme reverse transcriptase may be referred to as complementary DNA (cDNA). For this method, the products (ssDNA) are used as a template for the PCR. The PCR products are then measured, providing an indirect measurement of the products of the reverse transcription reaction. However, the method has disadvantages in that the method is time-consuming, and the products of the reverse transcription assay are indirectly measured. Further, even if a real-time PCR is performed, the results are converted to a cycle threshold (Ct) value, which does not provide a direct measurement of the products. Furthermore, since primers are used in the PCR, the accuracy of the measurement may vary according to the types of the primer.

Another conventional method of measuring products of the reverse transcription includes hybridizing products of the reverse transcription to probes immobilized on a microarray, and measuring the products based on the hybridization has been reported. However, the method is time-consuming and complicated. In addition, according to this method, the products of the reverse transcription are indirectly measured according to the method.

Thus, a method of efficiently measuring products of the reverse transcription has further scope for improvement.

### SUMMARY

Disclosed herein is a method of measuring a single stranded DNA reverse-transcribed from a RNA template.

One or more embodiments include method of measuring activity of a reverse transcriptase.

One or more embodiments include a kit for measuring a single stranded DNA reverse-transcribed from a RNA template.

One or more embodiments include a kit for measuring activity of a reverse transcriptase.

In one embodiment, a method of measuring a single stranded DNA reverse-transcribed from a RNA template includes: annealing a primer to the RNA template by incubating a sample including the RNA template with the primer; reverse-transcribing DNA from the RNA by incubating the annealed product in the presence of a reverse transcriptase and dNTP; incubating the reverse-transcribed product in the presence of RNaseH, RNaseT1, and RNase A; labeling a single stranded DNA by adding a DNA labeling material to the incubated product; and measuring a signal from the labeled single stranded DNA.

According to one or more embodiments of the present invention, a method of measuring activity of a reverse transcriptase includes calculating the amount of the produced single stranded DNA per reaction time based on the amount of single stranded DNA reverse-transcribed from the RNA template which is measured according to the above method of measuring a single stranded DNA reverse-transcribed from a RNA template.

According to one or more embodiments of the present invention, a kit for measuring a single stranded DNA reverse-transcribed from a RNA template includes a primer, a reverse transcriptase, dNTP, RNaseH, RNaseT1, RNase A, and a DNA labeling material.

According to one or more embodiments of the present invention, a kit for measuring activity of a reverse transcriptase includes a primer, a reverse transcriptase, dNTP, RNaseH, RNaseT1, RNase A, and a DNA labeling material.

### BRIEF DESCRIPTION OF THE DRAWINGS

The above and other aspects, advantages and features of this disclosure will become apparent and more readily appreciated from the following description of the embodiments, taken in conjunction with the accompanying drawings of which:

FIG. 1 is a graph illustrating the amount of DNA obtained by treating reverse transcribed products using RNase H, RNase T1, and RNase A having different concentrations, labeling ssDNA with Oligogreen™, as a ssDNA-specific fluorescent substance, and measuring fluorescence emitted therefrom; and the unit for the y-axis is fluorescence unit (FU).

FIG. 2 is a graph illustrating the amount of RNA obtained by treating reverse transcribed products using RNase H, RNase T1, and RNase A having different concentrations, labeling RNA with Ribogreen™, as a RNA-specific fluorescent substance, and measuring fluorescence emitted therefrom. The unit for the y-axis is fluorescence unit (FU).

### DETAILED DESCRIPTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Reference will now be made in detail to embodiments, examples of which are illustrated in the accompanying drawings.

As used herein, the term “dNTP” refers to deoxynucleoside triphosphates, the building blocks from which the DNA polymerases synthesizes a new DNA strand. dNTPs include 2′-deoxyadenosine-5′-triphosphate (dATP), 2′-deoxycytidine-5′-triphosphate (dCTP), 2′-deoxyguanosine-5′-triphosphate (dGTP), and 2′-deoxythymidine-5′-triphosphate (dTTP).

As used herein, the abbreviations “A,” “T,” “G,” “C” and “U” describe both the ribonucleotides and the deoxyribonucleotides. The abbreviations C, A and G are used to describe both the ribonucleotides and the deoxyribonucleotides, according to context. The abbreviation T is used to describe the deoxyribonucleotide. The abbreviation U is used to describe the ribonucleotide.

According to an embodiment, there is provided a method of measuring a single stranded DNA reverse-transcribed from a RNA template, the method including: annealing a primer to the RNA template by incubating a sample including the RNA template with the primer; reverse-transcribing DNA from the RNA by incubating the annealed product in the presence of a reverse transcriptase and dNTP; incubating the reverse-transcribed product in the presence of RNaseH, RNaseT1, and RNase A; labeling a single stranded DNA by adding a DNA labeling material to the incubated product; and measuring a signal from the labeled single stranded DNA.

The method includes annealing the primer to the RNA template by incubating the sample including the RNA template with the primer. The annealing may include denaturing the RNA template. The denaturing of the RNA template may be performed by thermal treatment, for example, by incubating at a temperature ranging from about 65° C. to about 75° C. The primer may be annealed to the denatured RNA template at a low temperature, for example, at a temperature ranging from about 0° C. to about 10° C.

The sample including the RNA template may further include other RNAs in addition to the template RNA. The sample may include mRNA and rRNA, or mRNA, rRNA and tRNA. The sample may be total cellular RNA isolated from a eukaryotic cell or a prokaryotic cell. The method of isolating the total cellular RNA is known in the art. For example, the total cellular RNA may be isolated using a Trizol reagent (e.g., TRizol™ Plus RNA Purification System: Invitrogen). The RNA template may be mRNA.

The primer may be selected from the group consisting of an oligo-dT primer, a random primer, and a gene-specific primer.

In one embodiment, the method includes reverse-transcribing DNA from the RNA by incubating the annealed product in the presence of a reverse transcriptase and dNTP. The incubation solution may further include an RNase inhibitor and a reverse transcriptase buffer. Conditions for the reverse transcriptase, such as the pH and temperature are well known in the art. The reverse-transcribing may include incubating the annealed product at a temperature ranging from about 37° C. to about 45° C., for example, at 42° C.

As used herein “reverse transcriptase” refers to a RNA-dependent DNA polymerase, which is a DNA polymerase enzyme that transcribes single stranded RNA into single-stranded DNA. As a result of the reverse-transcribing, a RNA/DNA duplex is produced. In one embodiment, the reverse transcriptase may be selected from the group consisting of M-MLV reverse transcriptase, AMV reverse transcriptase, HIV-1 reverse transcriptase, and telomerase reverse transcriptase.

In one embodiment, the method may further include inactivating the reverse transcriptase after the enzyme transcribes the RNA into single-stranded DNA. The inactivation of the reverse transcriptase may be performed by thermal treatment, for example, by heat-treating at 70° C.

In one embodiment, the method includes incubating the reverse-transcribed product in the presence of one or more ribonucleases. Ribonucleases include, for example, RNase H, RNase T1, and RNase A. RNase H is a ribonuclease that cleaves the 3′-O—P bond of RNA in a RNA/DNA duplex to produce 3′-hydroxy and 5′-phosphate terminated products. RNase H is a non-specific endonuclease and catalyses the cleavage of RNA via a hydrolytic mechanism. RNaseT1 is a fungal endonuclease that cleaves single stranded RNA after guanine residues, i.e., on their 3′ end. The most commonly studied form of this enzyme is the version encoded by rntA gene found in the mold Aspergillus oryzae. RNase A is an endonuclease cleaving the single stranded RNA, and may be bovine pancreatic RNase A. The RNase A cleaves RNA at C and U residues. Conditions for the RNaseH, RNaseT1, and RNase A are well known in the art, and the RNaseH, RNaseT1, and RNase A may compatibly catalyze the subject in the same condition. For example, the RNaseH, RNaseT1, and RNase A may be used at a temperature ranging from about 20° C. to about 40° C., for example, about 25° C. or about 37° C., but the temperature is not limited thereto. In addition, buffers appropriate for each of the RNaseH, RNaseT1, and RNase A may be compatibly used, or a reverse transcriptase buffer may be compatibly used. The incubating may be performed in the presence of a RNase H buffer (75 mM KCl, 50 mM Tris-HCl, 3 mM MgCl₂, 10 mM Dithiothreitol, pH 8.3, 25° C.), a RNase T1 buffer (with 100-200 mM salt (NaCl or KOAc), pH 7.0-8.8), a RNase A buffer, and a reverse transcription buffer. The incubating may be performed in the presence of the reverse transcriptase buffer or without buffers. The RNaseT1 and RNase A may be provided by a mixture of RNaseT1 and RNase A, e.g., RNase A/T1 Mix (Fermentas Inc. U.S.A.). As a result of the incubating, RNA of the RNA/DNA duplex and single stranded RNA, e.g., rRNA and tRNA, are removed.

The method includes labeling a single stranded DNA by adding a DNA labeling material to the incubated product. The DNA labeling material may be any labeling material that may label DNA without limitation. For example, the labeling material may be an intercalator into which DNA is intercalated. In addition, the labeling material may be a single stranded DNA-specific labeling material. The labeling material may be Oligogreen™ (Invitrogen), Cuproline blue, ethidium bromide (EtBr), and Ribogreen™, but is not limited thereto.

The method includes measuring a signal associated with the labeled single stranded DNA. The signal may be measured using a known method according to the types of the labeling material. For example, if the labeling material is Oligogreen™ (Invitrogen), an excitation light having a wavelength of about 480 nm is irradiated, and fluorescence is measured at about 520 nm. The measured signal represents the existence or the amount of the reverse-transcribed ssDNA.

In one embodiment, the method may further include a negative control group. For example, the method may further include: incubating the sample including the RNA template in the presence of RNase H, RNase T1, and RNase A; and measuring a signal from the incubated product and using the signal as a negative control group. For the negative control group, incubating the sample in the presence of RNase H, RNase T1, and RNase A is described above. In addition, the signal value detected in the negative control group may be used as an indicator of the background signal, where the signal value in the negative control group is subtracted from a signal value of an experimental group.

In one embodiment, the method may further include a positive control group. For example, the method may further include: labeling the single stranded DNA by adding a DNA labeling material, e.g., a single stranded DNA-specific labeling material, to a sample including ssDNA having a known concentration and sequence; and measuring a signal from the labeled single stranded DNA and using the signal as a positive control group.

According to another embodiment, there is provided a method of measuring the activity of a reverse transcriptase, the method including calculating the amount of single stranded DNA produced per reaction time based on the amount of single stranded DNA reverse-transcribed from the RNA template measured according to the method described above.

The reaction time indicates a time period from the initiation of the reaction of the reverse transcriptase to the termination of the reaction.

According to another embodiment, there is provided a kit for measuring a single stranded DNA reverse-transcribed from a RNA template, the kit including a primer, a reverse transcriptase, dNTP, Rnase H, Rnase T1, RNase A, and a DNA labeling material, e.g., a single stranded DNA-specific labeling material. The primer, the reverse transcriptase, dNTP, RNaseH, RNaseT1, RNase A, and the DNA labeling material are described above with reference to the method of measuring a single stranded DNA reverse-transcribed from a RNA template.

The kit may include a manual explaining how to use the kit according to the method of measuring a single stranded DNA reverse-transcribed from a RNA template disclosed herein.

According to another embodiment, there is provided a kit for measuring the activity of a reverse transcriptase, the kit including a primer, a reverse transcriptase, dNTP, RNase H, RNase T1, RNase A, and a DNA labeling material, e.g., a single stranded DNA-specific labeling material. The primer, the reverse transcriptase, dNTP, RNaseH, RNaseT1, RNase A, and the DNA labeling material are described above with reference to the method of measuring a single stranded DNA reverse-transcribed from a RNA template disclosed herein.

The kit may include a manual explaining how to use the kit according to the method of measuring the activity of the reverse transcriptase.

Hereinafter, one or more embodiments will be described in detail with reference to the following examples. However, these examples are not intended to limit the purpose and scope of the invention

### Example 1

### Influence of RNaseH, RNaseT1, and RNase A on Reverse-Transcribed Products

For this example, single stranded DNA was synthesized from universal human reference RNA (UHRR) in a reaction catalyzed by a reverse transcriptase enzyme. When the reverse transcription was terminated, the resultant was treated with RNaseH, RNaseT1, and RNase A to remove the RNA. Then, reverse-transcribed ssDNA was measured using a ssDNA-specific fluorescent substance.

### (1) RNA Sample

The universal human reference RNA (UHRR), which is purified human total cellular RNA, manufactured by Stratagene Inc. (Catalog no. 740000) was used as a RNA sample.

### (2) Reverse Transcription

1 μl of the RNA (1 μg/μL) was mixed with 50 pmole of a primer having SEQ ID NO: 1, and the mixture was heat-treated at 70° C. for 10 minutes. Then, the resultant mixture was rapidly transferred to an ice bath to anneal the primer to the RNA template. Then, a reverse transcription reaction was performed using a MessageAmp™ II-Biotin Enhanced Kit manufactured by Abion Corporation, according to its manual.

In more detail, the RNA was mixed with the primer, and water without nuclease was added thereto to adjust the volume of the mixture to 12 μl. Then, the mixture was incubated at 70° C. for 10 minutes. Next, the resultant mixture was transferred to an ice bath. Then, a reverse transcription master mix was prepared at room temperature in the absence of nuclease. The master mix was prepared by sequentially mixing 2 μl of 10× of a single stranded buffer, 4 μl of a dNTP mix, 1 μl of a RNase inhibitor, and 1 μl of ArrayScript. 8 μl of the master mix was added to the sample, bringing the total volume of the reverse transcription assay to 20 μl, and the mixture was incubated at 42° C. for 2 hours.

(3) Treatment with RNase and Labeling ssDNA with ssDNA-Specific Labeling Material

Following the 2 hour incubation, 10 units of RNase H, 100 units of RNase T1, and 10 units of RNase A were added to the products of the reverse transcription, and the mixture was incubated at 25° C. for 15 hours. Oligogreen™ (Invitrogen) was added to the mixture treated with the RNase H, RNase T1, and RNase A to label ssDNA. Then, an excitation light having a wavelength of about 480 nm was irradiated thereto, and fluorescence was measured at about 520 nm using a Qubit™ (Invitrogen).)

As an experimental group, Ribogreen™, which is a RNA-specific fluorescent substance, was added to mixture treated with the RNase H, RNase T1, and RNase A using a Ribogreen™ RNA quantification kit (Molecular Probes, Inc) according to its manual to label RNA. Then, an excitation light having a wavelength of about 480 nm was irradiated thereto, and fluorescence was measured at about 520 nm using a Qubit™ (Invitrogen).

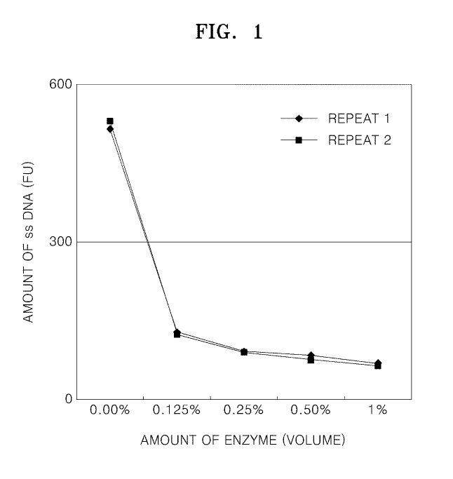

FIG. 1 is a graph illustrating the amount of DNA obtained by treating reverse transcribed products using RNase H, RNase T1, and RNase A having different concentrations, labeling ssDNA with Oligogreen™, as a ssDNA-specific fluorescent substance, and measuring fluorescence emitted therefrom. In FIG. 1, the x-axis indicates concentrations (%) of RNaseH, RNaseT1, and RNase A (volume of enzyme per volume of total incubated solution), and the y-axis indicates the amount of DNA measured at 520 nm. The unit for the y-axis indicating the amount of DNA measured at 520 nm is a fluorescence unit (FU). As shown in FIG. 1, the intensity of fluorescence significantly decreases when the concentration of RNase H, RNase T1, and RNase A changes from 0 to 0.125%. Oligogreen™ mainly binds with ssDNA with minor cross reaction with RNA molecule. Thus, the result of FIG. 1 indicates that fluorescence derived from a cross-reaction product between Oligogreen™ and RNA decreases since the DNA would not be affected by the treatment of RNase H, RNase T1, and RNase A. Thus, referring to FIG. 1, a large amount of RNA is contained in the products of the reverse transcription, and most of the RNA may be removed by treating with 0.125% to 1% of RNaseH, RNaseT1, and RNase A. That is, the amount of fluorescence derived from RNA constitutes noise when ssDNA is measured. The ssDNA may be calculated using negative control data and positive control experimental data. For example, the ssDNA may be calculated by subtracting the signal value from a negative control from that of the experimental group and converting the signal value to the amount of ssDNA by using a positive control data.

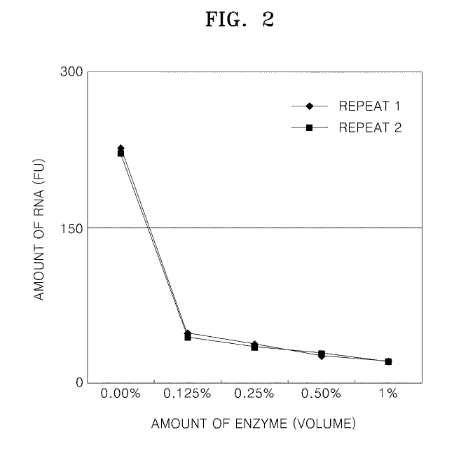

FIG. 2 is a graph illustrating the amount of RNA obtained by treating reverse transcribed products using RNase H, RNase T1, and RNase A having different concentrations, labeling RNA with Ribogreen™, as a RNA-specific fluorescent substance, and measuring fluorescence emitted therefrom. In FIG. 2, the x-axis indicates concentrations (%) of Rnase H, RNase T1, and RNase A (volume of enzyme per volume of total incubated solution), and the y-axis indicates the amount of RNA measured at 520 nm. The unit for the y-axis indicating the amount of RNA measured at 520 nm is fluorescence unit (FU). As shown in FIG. 2, the intensity of fluorescence significantly decreases when the concentration of RNase H, RNase T1, and RNase A changes from 0 to 0.125%. This indicates that fluorescence derived from a reaction product between Ribogreen™ and RNA decreases. Thus, referring to FIG. 2, a large amount of RNA is contained in the products of the reverse transcription, and most of the RNA may be removed by treating with 0.125% to 1% of RNase H, RNase T1, and RNase A. The detection limit for Qubit™ meter is about 20 FU.

According to the method of measuring the single stranded DNA reverse-transcribed from the RNA template, the single stranded DNA reverse-transcribed from the RNA template may be accurately, sensitively, and rapidly measured.

As described above, according to the one or more of the above embodiments, the single stranded DNA reverse-transcribed from a RNA template may be efficiently measured.

According to the one or more of the above embodiments, activity of a reverse transcriptase may be efficiently measured.

According to the one or more of the above embodiments, a kit for measuring a single stranded DNA reverse-transcribed from a RNA template may be efficiently used to measure a single stranded DNA reverse-transcribed from a RNA template.

According to the one or more of the above embodiments, the kit may be efficiently used to measure activity of a reverse transcriptase.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A method of measuring a single stranded DNA reverse-transcribed from a RNA template, the method comprising:
annealing a primer to the RNA template by incubating a sample comprising the RNA template with the primer;
reverse-transcribing DNA from the RNA by incubating the annealed product in the presence of a reverse transcriptase and dNTP;
incubating the reverse-transcribed product in the presence of RNase H, RNase T1, and RNase A;
labeling a single stranded DNA by adding a DNA labeling material to the incubated product; and
measuring a signal from the labeled single stranded DNA.

2. The method of claim 1, further comprising:
incubating the sample comprising the RNA template in the presence of RNaseH, RNaseT1, and RNase A; and
measuring a signal from the incubated product and using the signal as a negative control group.

3. The method of claim 1, wherein the sample comprises mRNA, rRNA and tRNA.

4. The method of claim 1, wherein the sample comprises total cellular RNA.

5. The method of claim 1, wherein the RNA template comprises mRNA.

6. The method of claim 1, wherein the primer comprises one selected from the group consisting of an oligo-dT primer, a random primer, and a gene-specific primer.

7. The method of claim 1, wherein the annealing comprises incubating the sample with the primer at a temperature ranging from about 65° to about 75°.

8. The method of claim 1, wherein the annealing comprises incubating the sample with the primer at a temperature ranging from about 0° to about 10°

9. The method of claim 1, wherein the reverse-transcribing comprises incubating the annealed product at a temperature ranging from about 37° to about 45°.

10. The method of claim 1, wherein the reverse transcriptase comprises one selected from the group consisting of M-MLV reverse transcriptase, AMV reverse transcriptase, HIV-1 reverse transcriptase, and telomerase reverse transcriptase.

11. The method of claim 1, wherein the DNA labeling material comprises one selected from the group consisting of Oligogreen™, Cuproline blue, and ethidium bromide (EtBr).

12. A method of measuring activity of a reverse transcriptase, the method comprising calculating the amount of single stranded DNA produced per reaction time based on the amount of single stranded DNA reverse-transcribed from the RNA template which is measured according to any one of claims 1.

13. A kit for measuring a single stranded DNA reverse-transcribed from a RNA template, the kit comprising a primer, a reverse transcriptase, dNTP, RNaseH, RNaseT1, RNase A, and a DNA labeling material.

14. A kit for measuring activity of a reverse transcriptase, the kit comprising a primer, a reverse transcriptase, dNTP, RNaseH, RNaseT1, RNase A, and a DNA labeling material.


SEQUENCE LISTING (1 sequence)

SEQ ID NO: 1 - DNA, 63 bp, Artificial Sequence
  PRIMER
ggccagtgaattgtaatacgactcactatagggaggcggtttttttttttttttttttttttt